SEQUENCE LISTING (2 sequences)

SEQ ID NO: 1 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 2 - DNA, 909 bp, Homo sapiens
acaccactgtttgtgctggatcgcagcgctgcctttccttatgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgcagccagctcccttaggaatgacagcagtagcagtaataggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaattgtggcttgtatcaacactgttactttcgtacattggctggtaaca

(19) United States
(12) Patent Application Publication
(10) Pub. No.: US 2015/0196622 A1
(43) Pub. Date: Jul. 16, 2015

(54) MATERIALS AND METHODS FOR MODULATING GLUCOSE UPTAKE

(71) Applicant: Ali Nayer, Miami, FL (US)

(72) Inventor: Ali Nayer, Miami, FL (US)

(21) Appl. No.: 14/415,762

(22) PCT Filed: Aug. 21, 2013
(86) PCT No.: PCT/US2013/055979

§ 371 Date: Jan. 20, 2015

Related U.S. Application Data
(60) Provisional application No. 61/691,628, filed on Aug. 21, 2012.

(51) Int. Cl. A61K 38/19; A61K 45/06

(52) U.S. Cl. CPC A61K 38/19; A61K 45/06

(57) ABSTRACT

The subject invention provides materials and methods for modulating cellular glucose uptake. Specifically, in accordance with one embodiment of the subject invention, stem cell factor (SCF) can be used to stimulate cellular glucose uptake. The administration of SCF can also be used to promote Glucose Transporter 4 (GLUT4) expression. In accordance with the subject invention, SCF can be used to improve glucose homeostasis, including in the treatment of diabetes mellitus.

### BACKGROUND OF INVENTION

Glucose is a vital source of energy for most organisms. The binding of insulin to its tyrosine kinase receptor promotes cellular glucose uptake and metabolism. Since cell membranes are impermeable to carbohydrates, glucose is taken up via membrane proteins known as glucose transporters. The glucose transporter 4 (GLUT4) accounts for 90% of glucose transporters in skeletal muscle and adipose tissue and mediated insulin stimulated glucose uptake.

Diabetes is a group of diseases characterized by high blood glucose levels, which result from defects in insulin production, insulin action, or both. Because diabetes can remain undiagnosed for years, many people become aware that they have diabetes only after the development of one of its life-threatening complications.

Diabetes is a leading cause of new cases of blindness in adults ages 20-74. Diabetes is also a leading cause of end-stage renal disease. About 60-70 percent of people with diabetes have mild to severe forms of diabetic nerve damage, which, in severe forms, can lead to lower limb amputations. In fact, more than 60% of non-traumatic, lower limb amputations are performed on persons with diabetes.

Diabetes is estimated to cost the U.S. health care system over 100 billion annually. More serious than the economic costs associated with diabetes are the decrease in quality of life, serious health complications/consequences, and deaths associated with diabetes.

Type 1 (or insulin-dependent diabetes mellitus or juvenile-onset diabetes), develops when the body's immune system destroys pancreatic cells that make the hormone insulin, which regulates blood glucose levels. Type 1 diabetes usually occurs in children and young adults; although disease onset can occur at any age. Type 1 diabetes accounts for about 5 to 10 percent of all diagnosed cases of diabetes. Individuals diagnosed with Type I diabetes typically require daily delivery of insulin via injections or pumps.

Type 2 diabetes (or non-insulin-dependent diabetes mellitus or adult-onset diabetes), is a metabolic disorder resulting from the body's inability to make enough, or properly use, insulin. This disease usually begins as insulin resistance, a disorder in which the cells do not use insulin properly, and as the need for insulin rises, the pancreas gradually loses its ability to produce insulin. Type 2 diabetes is the most common form of the disease accounting for 90-95 percent of diabetes. Type 2 diabetes is nearing epidemic proportions, due to an increased number of older Americans, and a greater prevalence of obesity and a sedentary lifestyle.

Gestational diabetes refers to a form of glucose intolerance that occurs in pregnant women. During pregnancy, gestational diabetes requires treatment to normalize maternal blood glucose levels to avoid complications in the infant. A percentage (5-10 percent) of women with gestational diabetes have Type 2 diabetes after pregnancy. Women who have had gestational diabetes also have a 20-50 percent chance of developing diabetes in the next 5-10 years.

Hyperinsulinemia refers to the overproduction of insulin by pancreatic cells. Often, hyperinsulinemia occurs as a result of insulin resistance, which is a condition defined by cellular resistance to the action of insulin. Insulin resistance is a state/disorder in which a normal amount of insulin produces a subnormal biologic (metabolic) response. For example, in insulin-treated patients with diabetes, insulin resistance is considered to be present whenever the therapeutic dose of insulin exceeds the secretory rate of insulin in normal person.

Impaired glucose homeostasis (or metabolism) refers to a condition in which blood sugar levels are higher than normal but not high enough to be classified as diabetes. There are two categories that are considered risk factors for future diabetes and cardiovascular disease. Impaired glucose tolerance (IGT) occurs when the glucose levels following a 2-hour oral glucose tolerance test are between 140 to 199 mg/dl. IGT is a major risk factor for type 2 diabetes and is present in about 11 percent of adults, or approximately 20 million Americans. About 40-45 percent of persons age 65 years or older have either type 2 diabetes or IGT. Impaired fasting glucose (IFG) occurs when the glucose levels following an 8-hour fasting plasma glucose test are greater than 110 but less than 126 mg/dl.

Insulin resistance is not primarily caused by a diminished number of insulin receptors but rather by a post-insulin receptor binding defect that is not yet completely understood. This lack of responsiveness to insulin results in insufficient insulin-mediated activation of uptake, oxidation and storage of glucose in muscle, and inadequate insulin-mediated repression of lipolysis in adipose tissue and of glucose production and secretion in the liver. Eventually, a patient may be become diabetic due to the inability to properly compensate for insulin resistance. In humans, the beta cells within the pancreatic islets initially compensate for insulin resistance by increasing insulin output.

Patients who have insulin resistance often exhibit several symptoms that together are referred to as Syndrome X or Metabolic Syndrome. Patients with Metabolic Syndrome have an increased risk of developing atherosclerosis and coronary heart disease.

AMP-activated protein kinase (AMPK) has been identified as a regulator of carbohydrate and fatty acid metabolism that helps maintain energy balance in response to environmental and nutritional stress. There is evidence that activation of AMPK results in a number of beneficial effects on lipid and glucose metabolism by reducing glucogenesis and de novo lipogenesis (fatty acid and cholesterol synthesis), and by increasing fatty acid oxidation and skeletal muscle glucose uptake. Inhibition of ACC, by phosphorylation by AMPK, leads to a decrease in fatty acid synthesis and to an increase in fatty acid oxidation, while inhibition of HMG-CoA reductase, by phosphorylation by AMPK, leads to a decrease in cholesterol synthesis (Carling, D. et al., FEBS Letters 223:217 (1987)).

In vivo activation of AMPK in the liver can result in the reduction of hepatic glucose output, an improvement in overall glucose homeostasis, a decrease in fatty acid and cholesterol synthesis, and an increase in fatty acid oxidation. Stimulation of AMPK in skeletal muscle can result in an increase in glucose uptake and fatty acid oxidation with resulting improvement of glucose homeostasis, and an improvement in insulin action. Finally, the resulting increase in energy expenditure should lead to a decrease in body weight. The lowering of blood pressure has also been reported to be a consequence of AMPK activation.

Increased fatty acid synthesis is a characteristic of many tumor cells; therefore, decreasing the synthesis of fatty acids via AMPK activation may also be useful as a cancer therapy. Activation of AMPK may also be useful to treat ischemic events in the brain (Blazquez, C. et. al., J. Neurochem. 73: 1674 (1999)); to prevent damage from reactive oxygen species (Zhou, M. et al., Am. J. Physiol. Endocrinol. Metab. 279: E622 (2000)); and to improve local circulatory systems (Chen, Z.-P., et. al. AMP-activated protein kinase phosphorylation of endothelial NO synthase. FEBS Letters 443: 285 (1999)).

Compounds that activate AMPK are expected to be useful to treat type 2 diabetes mellitus, obesity, hypertension, dyslipidemia, cancer, and metabolic syndrome, as well as cardiovascular diseases, such as myocardial infarction and stroke, by improving glucose and lipid metabolism and by reducing body weight. There is a need for potent AMPK activators that have pharmacokinetic and pharmacodynamic properties suitable for use as human pharmaceuticals.

Persistent or uncontrolled hyperglycemia is associated with increased and premature morbidity and mortality. Often abnormal glucose homeostasis is associated both directly and indirectly with obesity, hypertension, and alterations of the lipid, lipoprotein and apolipoprotein metabolism, as well as other metabolic and hemodynamic disease. Patients with Type 2 diabetes have a significantly increased risk of macrovascular and microvascular complications, including atherosclerosis, coronary heart disease, stroke, peripheral vascular disease, hypertension, nephropathy, neuropathy, and retinopathy. Therefore, effective therapeutic control of glucose homeostasis, lipid metabolism, obesity, and hypertension are critically important in the clinical management and treatment of diabetes.

Physical exercise and a reduction in dietary intake of calories often dramatically improve the diabetic condition and are the usual recommended first-line treatment of Type 2 diabetes and of pre-diabetic conditions associated with insulin resistance. Compliance with this treatment is generally very poor because of well-entrenched sedentary lifestyles and excess food consumption, especially of foods containing high amounts of fat and carbohydrates.

Pharmacologic treatments for diabetes have largely focused on six areas of pathophysiology: (1) hepatic glucose production (biguanides, such as phenformin and metformin), (2) insulin resistance (PPAR agonists, such as rosiglitazone, troglitazone, engliazone, balaglitazone, MCC-555, netoglitazone, T-131, LY-300512, LY-818 and pioglitazone), (3) insulin secretion (sulfonylureas, such as tolbutamide, glipizide and glimipiride); (4) incretin hormone mimetics (GLP-1 derivatives and analogs, such as exenatide and liraglitide); (5) inhibitors of incretin hormone degradation (DPP-4 inhibitors, such as sitagliptin); and (6) insulin replacement therapy.

Many of the current treatments for diabetes have unwanted side effects. Phenformin and metformin can induce lactic acidosis, nausea/vomiting, and diarrhea. Metformin has a lower risk of side effects than phenformin and is widely prescribed for the treatment of Type 2 diabetes. The currently marketed PPAR gamma agonists are modestly effective in reducing plasma glucose and hemoglobin A1C, and do not greatly improve lipid metabolism or the lipid profile. Sulfonylureas and related insulin secretagogues can cause insulin secretion even if the glucose level is low, resulting in hypoglycemia, which can be fatal in severe cases.

Stem cell factor (“SCF”) is a ligand for the tyrosine kinase receptor known as c-kit. See Zsebo, Cell 1990, 63 (1) 213-24); U.S. Pat. No. 6,218,148. SCF induces the proliferation of primitive CD34+ bone marrow progenitors. It has been shown in a number of studies in mice, dogs, and humans, that the SCF/c-kit pathway is involved in the normal process of repair following ischemic damage. SCF has also been called kit-ligand (PCT Pub. No. WO 92/03459), mast cell growth factor (PCT Pub. WO 92/00376) and Steel factor (White, Cell 63:5-6, 1990). The gene encoding SCF has been cloned and expressed, e.g., Martin et al. (Cell 63:203-211, 1990), and PCT Pub. No. WO 91/05795, which is herein incorporated by reference in its entirety.

SCF has numerous active forms, including a membrane bound version and a soluble version. See PCT Pub. No. 91/05795. C-terminal deletion analogs also have activity. For example, SCF 1-137 (with “1” referring to the first amino acid of the mature protein) demonstrates biological activity, and SCF 1-141 demonstrates more or less full biological activity (Langley et al., Arch. Biochem. Biophys. 311:55-61, 1994). SCF 1-165 having an aspartic acid at position 10, instead of an asparagine as in the native sequence (referred to as “N10D”) has also been studied, and found to not appreciably influence the rate of dimer formation (Lu et al., Biochem. J. 305: 563-568, 1995). Certain covalently linked SCF dimers are reported in PCT publication WO 95/26199.

U.S. Pat. No. 6,723,561 describes retroviral vectors encoding SCF and retroviral packaging cell lines expressing SCF and use of same to deliver a foreign nucleic acid to stem cells in a subject.

SCF has never before been reported to play a role in glucose metabolism. There remains a need for treatments for diabetes that work by novel mechanisms of action.

### BRIEF SUMMARY

The subject invention provides materials and methods for modulating cellular glucose uptake. Specifically, in accordance with one embodiment of the subject invention, stem cell factor (SCF) can be used to stimulate cellular glucose uptake. The administration of SCF can also be used to promote Glucose Transporter 4 (GLUT4) expression. In accordance with the subject invention, SCF can be used to improve glucose homeostasis, including in the treatment of diabetes mellitus.

In one embodiment of the subject invention, a composition comprising SCF, alone or in combination with one or more other active agents, is administered to a patient in need of improved cellular glucose uptake. The specific SCF formulations for such a therapy may be selected based on the route of administration.

SCF can be administered to one or more sites within the patient in a therapeutically effective amount. In certain embodiments, the SCF protein-based therapy is effected via continuous or intermittent intravenous administration.

In one embodiment of the subject invention, the administration of SCF is achieved via the expression of SCF from a cell that contains a polynucleotide encoding SCF. The cell can be a recombinant cell that has been transformed with DNA encoding SCF. The recombinant cell that expresses SCF can be a patient's own cell that has been transformed in vivo by the administration of a polynucleotide encoding SCF, or the SCF-expressing cell can be prepared ex vivo and administered to one or more specific locations where enhanced glucose uptake is desired.

In a further embodiment, the subject invention provides materials and methods for inhibiting cellular glucose uptake. This can be accomplished by, for example, blocking the activity of SCF and/or by blocking the c-kit receptor. The inhibition of glucose uptake that is achieved in this embodiment of the subject invention can be used, for example, to protect against low blood glucose concentrations (hypoglycemia).

The present invention also provides methods and compositions for the treatment, control or prevention of disorders, diseases, and conditions responsive to activation of AMP-activated protein kinase, in a subject in need thereof, by administering SCF.

The subject invention further contemplates combination therapies wherein SCF is administered to a patient in conjunction with another therapeutic agent. The other therapeutic agent may be, for example, an immuno-suppressive agent.

The present invention also provides pharmaceutical compositions comprising SCF, a pharmaceutically acceptable carrier and, optionally, additional active agents.

### BRIEF DESCRIPTION OF DRAWINGS

FIG. 1 shows that Western blot analysis of extracts from skeletal muscle, inguinal and epididymal fat of C57BL/6 mice demonstrate the expression of c-Kit and SCF in these tissues.

FIG. 2 shows immunostained samples of subcutaneous abdominal fat and rectus abdominis muscle. Confocal microscopy revealed diffuse staining for c-Kit in both adipose tissue and skeletal muscle (FIGS. 2A,B). Also, mast cells accounted for only a fraction of c-Kit immunoreactivity in these tissues (FIG. 2A). In addition, there was a diffuse and prominent immunoreactivity for SCF in adipose tissue and skeletal muscle samples obtained from humans (FIGS. 2C,D).

FIG. 3 shows that GLUT4 protein was expressed at significantly higher levels in inguinal (subcutaneous) than in epididymal (visceral) fat in C57BL/6 mice. This was accompanied by a greater SCF and c-Kit protein expression in inguinal than in epididymal fat in C57BL/6 mice (*P<0.05).

FIG. 4 shows that variation in GLUT4 protein expression was observed in inguinal (subcutaneous) fat of C57BL/6 mice of the same age and gender (FIG. 4A). Of note, there was a direct correlation between the expression levels of c-Kit and GLUT4 (FIG. 4B). In addition, there was a trend for an inverse correlation between c-Kit expression and blood glucose concentration in these mice (FIG. 4C).

FIG. 5 shows that, thirty minutes after injection, mice treated with 1.25 μg recombinant SCF had a lower blood glucose concentration than those treated with vehicle (203±3 vs. 235±10 mg/dl, P<0.05). A further decline in blood glucose concentration was observed when mice were treated with 2.5 μg of recombinant SCF (176±9 mg/dl, P<0.01).

FIG. 6 shows that, on an equimolar basis, SCF was as potent as insulin in promoting glucose uptake in 3T3-L1 adipocytes (FIG. 6A). In C2C12 myotubes, SCF stimulation led to an even greater increase in glucose uptake than insulin (FIG. 6B) (*P<0.05, **P<0.0).

FIG. 7 shows that, while insulin stimulation resulted in phosphorylation of insulin receptor β chain at tyrosine cluster 1146/1150/1151, none of these residues were phosphorylated upon SCF stimulation of 3T3-L1 adipocytes (FIG. 7A). Also insulin, but not SCF, resulted in phosphorylation of tyrosine 1150/1151 of the insulin receptor 3 chain in C2C12 myotubes (FIG. 7A). Also, stimulation of 3T3-L1 adipocytes and C2C12 myotubes with SCF resulted in a dose-dependent phosphorylation of the a catalytic subunit of AMPK at Thr172 residue (FIG. 7B). This phosphorylation is required for AMPK activation. In addition, SCF treatment of C2C12 myotubes resulted in phosphorylation of serine 108 residue of the β1 regulatory subunit of AMPK (FIG. 7B).

FIG. 8 shows that SCF treatment resulted in increased GLUT4 protein expression in 3T3-L1 adipocytes and C2C12 myotubes.

FIG. 9 shows that, compared with KitW/Kit+, KitWν/Kit+, and wild-type littermates (Kit+/Kit+), mice with loss-of-function mutations affecting both c-kit alleles (KitW/KitWν) demonstrated increased fasting blood glucose concentration despite a smaller body mass (FIGS. 9A-H). Male and female mice 4 weeks (FIGS. 9A-D) and 6 weeks (FIGS. 9E-H) of age were studied.

FIG. 10 shows that KitW/KitWν mice demonstrated increased fasting glucose and serum insulin concentrations resulting in a greater insulin resistance index, as determined by the homeostasis model assessment of insulin resistance (HOMA-IR) (FIGS. 10A-C). Similarly, compared with wildtype littermates, KitW/KitWν mice demonstrated attenuated glucose disposal in response to insulin (FIG. 10D). Furthermore, the glucose tolerance test revealed that KitW/KitWν mice disposed of a glucose load less efficiently than wild-type controls did (FIG. 10E).

FIG. 11 shows that, compared with wild-type controls, KitW/KitWν mice demonstrated a greater inguinal fat mass when adjusted for body weight (FIGS. 11A,B). Whereas there was also a trend for a greater adjusted epididymal fat mass, adjusted liver mass did not differ between KitW/KitWν mice and wild-type controls (FIG. 11C-D). Also, compared with wild-type controls, GLUT4 expression was attenuated in inguinal (subcutaneous) fat, epididymal (visceral) fat, and gastrocnemius muscle from KitW/KitWν mice (FIG. 11E).

FIG. 12 is the native amino acid sequence of the full length human SCF (SEQ ID NO:1).

FIG. 13 shows a nucleic acid sequence encoding a full length human SCF. The methionine start codon is shown with emphasis starting at nucleotide position 41 and the stop codon TAA is shown with emphasis beginning at position nucleotide 860 (SEQ ID NO:2).

### DETAILED DISCLOSURE

The subject invention provides materials and methods for modulating cellular glucose uptake. Specifically, in accordance with one embodiment of the subject invention, stem cell factor (SCF) can be used to stimulate cellular glucose uptake. The administration of SCF can also be used to promote Glucose Transporter 4 (GLUT4) expression. In accordance with the subject invention, SCF can be used to improve glucose homeostasis, including in the treatment of diabetes mellitus.

In accordance with the subject invention SCF and c-Kit proteins have been found to be expressed in adipose tissue and skeletal muscle in mice and humans. c-Kit expression in adipose tissue was found to correlate inversely with blood glucose concentrations and directly with GLUT4 protein expression. Furthermore, SCF, c-Kit, and GLUT4 expression levels were greater in subcutaneous than in visceral fat in mice.

SCF administration to mice resulted in an acute and dose-dependent decline in blood glucose concentration. Interestingly, SCF enhanced glucose uptake in cultured adipocytes and myocytes independent of the insulin receptor. Moreover, SCF stimulation resulted in activating phosphorylation of AMP-activated protein kinase, an evolutionary conserved sensor of cellular energy status involved in cellular glucose uptake and GLUT4 expression.

Compared with congenic wild-type controls, c-kit knockout mice showed systemic insulin resistance as indicated by higher fasting blood glucose and serum insulin concentrations, greater insulin resistance index, and attenuated glucose disposal in response to insulin. In addition, c-kit knockout mice demonstrated diminished GLUT4 protein expression in adipose tissue and skeletal muscle. SCF treatment increased the expression of GLUT4 in cultured adipocytes and myocytes.

One of the therapeutic embodiments of the present invention is the administration, to a subject in need thereof, of compositions comprising SCF, alone or in combination with other active agents. The SCF formulations for such a therapy may be prepared based on the route of administration.

Any route known to those of skill in the art for the administration of a therapeutic composition of the invention is contemplated including, for example, intravenous, intramuscular, subcutaneous or a catheter for long-term administration. Administration of the compositions can be systemic or local, and may comprise a single site or multiple site injection of a therapeutically-effective amount of the SCF. The multiple administrations may be rendered simultaneously or may be administered over a period of several hours. In certain cases, it may be beneficial to provide a continuous flow of the therapeutic composition. Additional therapy may be administered on a periodic basis, for example, daily, weekly, or monthly.

In one embodiment of the subject invention, the administration of SCF is achieved via the expression of SCF from a cell that contains a polynucleotide encoding SCF. The cell can be a recombinant cell that has been transformed with DNA encoding SCF. The recombinant cell that expresses SCF can be a patient's own cell that has been transformed in vivo by the administration of a polynucleotide encoding SCF, or the SCF-expressing cell can be prepared ex vivo and administered to one or more specific locations where enhanced glucose uptake is desired. The SCF-expressing cell can be located at, or administered to, for example, tissue that is particularly susceptible to disruptions in glucose homeostatis and/or locations where excessive insulin levels cause or contribute to pathological conditions. These locations include, but are not limited to, diabetic ulcers and the eyes of individuals having, or susceptible to, diabetic retinopathy.

In one embodiment, in order to reduce the likelihood of rejection by the immune system, SCF-expressing cells that are prepared ex vivo originate from the patient to be treated or from a genetically identical patient. Alternatively, the cells can be modified to reduce their immunogenicity. In one embodiment, these cells are genetically modified (SCF-overexpressing) stem cells located in, or isolated from, the patient's own tissue, such as fat, which is reported to have large numbers of stem cells.

In a further embodiment, the subject invention provides materials and methods for inhibiting cellular glucose uptake. This can be accomplished by, for example, blocking the activity of SCF and/or by blocking the c-Kit receptor. In specific embodiments, antibodies can be used to inhibit the activity of SCF and/or the c-Kit receptor. Alternatively, an SCF analog that binds to, but does not activate, the c-Kit receptor can be used. Inhibitors of the c-Kit receptor are also described in, for example, U.S. Pat. Nos. 7,915,391; 7,947,708; and 7,994,159. The inhibition of glucose uptake that is achieved in this embodiment of the subject invention can be used to protect against low blood glucose concentrations (hypoglycemia). Hypoglycemia can lead to brain damage as the brain is dependent on glucose as a source of fuel. Hypoglycemia is a common side effect of insulin therapy for diabetes mellitus.

The present invention also provides methods for the treatment, control or prevention of disorders, diseases, and conditions responsive to activation of AMP-activated protein kinase in a subject in need thereof by administering SCF and pharmaceutical compositions of the present invention. The present invention also relates to the use of SCF for manufacture of a medicament useful in treating diseases, disorders and conditions responsive to the activation of AMP-activated protein kinase. The present invention is also concerned with treatment of these diseases, disorders and conditions by administering SCF in combination with a therapeutically effective amount of another agent known to be useful to treat the disease, disorder and condition.

The subject invention further contemplates combination therapies wherein SCF is administered to a patient in conjunction with another therapeutic agent. The other therapeutic agent may be, for example, an immuno-suppressive agent.

The present invention also relates to pharmaceutical compositions comprising SCF, a pharmaceutically acceptable carrier and, optionally, additional active agents.

### Selected Definitions

With reference to the materials and methods of the present invention, the following terms are used with the noted meanings:

The terms “treating” or “treatment” of a disease includes:

(1) reducing the risk of developing the disease, e.g., causing the clinical symptoms of the disease not to develop in a mammal that may be predisposed to the disease but does not yet experience or display symptoms of the disease,

(2) inhibiting the disease, e.g., arresting or reducing the development of the disease or its clinical symptoms, or

(3) relieving the disease, e.g., causing regression of the disease or its clinical symptoms.

The term “diagnosing” refers to determining the presence or absence of a particular disease or condition. Additionally, the term refers to determining the level or severity of a particular disease or condition, as well as monitoring of the disease or condition to determine its response to a particular therapeutic regimen.

The term “therapeutically effective amount” means the amount of a compound that will elicit a desired biological or medical response. “A therapeutically effective amount” includes the amount of a compound that, when administered to a mammal for treating a disease, is sufficient to effect such treatment for the disease.

“Insulin resistance” is a disorder of glucose metabolism. More specifically, insulin resistance can be defined as the diminished ability of insulin to exert its biological action across a broad range of concentrations producing less than the expected biologic effect (see, e.g., Reaven, G. M. J. Basic & Clin. Phys. & Pharm. (1998) 9: 387-406 and Flier, J. Ann Rev. Med. (1983) 34: 145-60). Insulin resistant persons have a diminished ability to properly metabolize glucose and respond poorly, if at all, to insulin therapy. Manifestations of insulin resistance include insufficient insulin activation of glucose uptake, oxidation and storage in muscle and inadequate insulin repression of lipolysis in adipose tissue and of glucose production and secretion in liver. Insulin resistance can cause or contribute to polycystic ovarian syndrome, impaired glucose tolerance, gestational diabetes, metabolic syndrome, hypertension, obesity, atherosclerosis and a variety of other disorders. Eventually, the insulin resistant individuals can progress to a point where a diabetic state is reached.

The term “diabetes mellitus” or “diabetes” means a disease or condition that is generally characterized by metabolic defects in production and utilization of glucose that result in the failure to maintain appropriate blood sugar levels in the body. The result of these defects is elevated blood glucose, referred to as “hyperglycemia.” Two major forms of diabetes are Type I diabetes and Type II diabetes. As described above, Type I diabetes is generally the result of an absolute deficiency of insulin, the hormone that regulates glucose utilization. Type II diabetes often occurs in the presence of normal, or even elevated levels of insulin and can result from the inability of tissues to respond appropriately to insulin. Most Type II diabetic patients are insulin resistant and have a relative deficiency of insulin, in that insulin secretion cannot compensate for the resistance of peripheral tissues to respond to insulin. In addition, many Type II diabetics are obese. Other types of disorders of glucose homeostasis include impaired glucose tolerance, which is a metabolic stage intermediate between normal glucose homeostasis and diabetes, and gestational diabetes mellitus, which is glucose intolerance in pregnancy in women with no previous history of Type I or Type II diabetes.

The term “metabolic syndrome” refers to a cluster of metabolic abnormalities including abdominal obesity, insulin resistance, glucose intolerance, diabetes, hypertension and dyslipidemia. These abnormalities are known to be associated with an increased risk of vascular events.

The term “abdominal obesity” is defined by a cutoff point of waist circumference of 102 cm in men and 80 cm in women, as recommended by the third report of the national cholesterol education program expert panel on detection, evaluation, and treatment of high blood cholesterol in adults (NCEP/ATP Panel III).

The guidelines for diagnosis of Type II diabetes, impaired glucose tolerance, and gestational diabetes have been outlined by the American Diabetes Association (see, e.g., The Expert Committee on the Diagnosis and Classification of Diabetes Mellitus, Diabetes Care, (1999) Vol 2 (Suppl 1): S5-19).

The term “symptom” of diabetes, includes, but is not limited to, polyuria, polydipsia, and polyphagia, as used herein, incorporating their common usage. For example, “polyuria” means the passage of a large volume of urine during a given period; “polydipsia” means chronic, excessive thirst; and “polyphagia” means excessive eating. Other symptoms of diabetes include, e.g., increased susceptibility to certain infections (especially fungal and staphylococcal infections), nausea, and ketoacidosis (enhanced production of ketone bodies in the blood).

The term “complication” of diabetes includes, but is not limited to, microvascular complications and macrovascular complications. Microvascular complications are those complications that generally result in small blood vessel damage. These complications include, e.g., retinopathy (the impairment or loss of vision due to blood vessel damage in the eyes); neuropathy (nerve damage and foot problems due to blood vessel damage to the nervous system); and nephropathy (kidney disease due to blood vessel damage in the kidneys). Macrovascular complications are those complications that generally result from large blood vessel damage. These complications include, e.g., cardiovascular disease and peripheral vascular disease. Cardiovascular disease refers to diseases of blood vessels of the heart. See. e.g., Kaplan, R. M. et al., “Cardiovascular diseases” in HEALTH AND HUMAN BEHAVIOR, pp. 206-242 (McGraw-Hill, New York 1993). Peripheral vascular disease refers to diseases of any of the blood vessels outside of the heart. It is often a narrowing of the blood vessels that carry blood to leg and arm muscles.

The term “modulate” refers to the treating, prevention, suppression, enhancement or induction of a function or condition. For example, compounds can modulate Type II diabetes by increasing insulin in a human, thereby suppressing hyperglycemia.

The term “triglyceride(s)” (“TGs”), as used herein, incorporates its common usage. TGs consist of three fatty acid molecules esterified to a glycerol molecule. TGs serve to store fatty acids that are used by muscle cells for energy production or are taken up and stored in adipose tissue.

The term “dyslipidemia” refers to abnormal levels of lipoproteins in blood plasma including both depressed and/or elevated levels of lipoproteins (e.g., elevated levels of LDL and/or VLDL, and depressed levels of HDL).

The term “hyperlipidemia” includes, but is not limited to, the following:

(1) Familial Hyperchylomicronemia, a rare genetic disorder that causes a deficiency in an enzyme, LP lipase, that breaks down fat molecules. The LP lipase deficiency can cause the accumulation of large quantities of fat or lipoproteins in the blood;

(2) Familial Hypercholesterolemia, a relatively common genetic disorder caused where the underlying defect is a series of mutations in the LDL receptor gene that result in malfunctioning LDL receptors and/or absence of the LDL receptors. This brings about ineffective clearance of LDL by the LDL receptors resulting in elevated LDL and total cholesterol levels in the plasma;

(3) Familial Combined Hyperlipidemia, also known as multiple lipoprotein-type hyperlipidemia; an inherited disorder where patients and their affected first-degree relatives can at various times manifest high cholesterol and high triglycerides. Levels of HDL cholesterol are often moderately decreased;

(4) Familial Defective Apolipoprotein B-100 is a relatively common autosomal dominant genetic abnormality. The defect is caused by a single nucleotide mutation that produces a substitution of glutamine for arginine, which can cause reduced affinity of LDL particles for the LDL receptor. Consequently, this can cause high plasma LDL and total cholesterol levels;

(5) Familial Dysbetalipoproteinemia also referred to as Type III Hyperlipoproteinemia, is an uncommon inherited disorder resulting in moderate to severe elevations of serum TG and cholesterol levels with abnormal apolipoprotein E function. HDL levels are usually normal; and

(6) Familial Hypertriglyceridemia, is a common inherited disorder in which the concentration of plasma VLDL is elevated. This can cause mild to moderately elevated TG levels (and usually not cholesterol levels) and can often be associated with low plasma HDL levels.

Risk factors for hyperlipidemia include, but are not limited to, the following: (1) disease risk factors, such as a history of Type I diabetes, Type II diabetes, Cushing's syndrome, hypothyroidism and certain types of renal failure; (2) drug risk factors, which include, birth control pills; hormones, such as estrogen, and corticosteroids; certain diuretics; and various beta blockers; (3) dietary risk factors include dietary fat intake per total calories greater than 40%; saturated fat intake per total calories greater than 10%; cholesterol intake greater than 300 mg per day; habitual and excessive alcohol use; and obesity.

The terms “obese” and “obesity” refer to, according to the World Health Organization, a Body Mass Index (“BMI”) greater than 27.8 kg/m² for men and 27.3 kg/m² for women (BMI equals weight (kg)/height (m²). Obesity is linked to a variety of medical conditions including diabetes and hyperlipidemia. Obesity is also a known risk factor for the development of Type II diabetes (See, e.g., Barrett-Conner, E. Epidemol. Rev. (1989) 11: 172-181; and Knowler, et al. Am. J. Clin. Nutr. (1991) 53:1543-1551).

The term “insulin” refers to a polypeptide hormone that regulates glucose metabolism. Insulin binds to insulin receptors in insulin sensitive cells and mediates glucose uptake. Insulin is used to treat type 1 diabetes and may be used to treat type II diabetes.

The term “cAMP” or “cyclic AMP” or “cyclic adenosine monophosphate” refers to an intracellular signaling molecule involved in many biological processes, including glucose and lipid metabolism.

The term “agonist” refers to a compound that binds to a receptor and triggers a response in a cell. An agonist mimics the effect of an endogenous ligand, a hormone for example, and produces a physiological response similar to that produced by the endogenous ligand.

The term “partial agonist” refers to a compound that binds to a receptor and triggers a partial response in a cell. A partial agonist produces only a partial physiological response of the endogenous ligand.

### Stem Cell Factor

The term “stem cell factor” or “SCF” as used herein refers to naturally-occurring SCF (e.g. natural human-SCF) as well as non-naturally occurring (i.e., different from naturally occurring) polypeptides having amino acid sequences and glycosylation sufficiently duplicative of that of naturally-occurring stem cell factor to facilitate possession of a hematopoietic biological activity of naturally-occurring stem cell factor.

Stem cell factor has the ability to stimulate growth of carly hematopoietic progenitors, which are capable of maturing to erythroid, megakaryocyte, granulocyte, lymphocyte, and macrophage cells. SCF treatment of mammals results in increases in hematopoietic cells of both myeloid and lymphoid lineages. One of the hallmark characteristics of stem cells is their ability to differentiate into both myeloid and lymphoid cells (Spangrude, Science 241:58-62, 1988).

In addition to naturally-occurring allelic forms of SCF, the present invention also embraces other SCF products such as polypeptide analogs of SCF. Such analogs include fragments of SCF. According to the procedures of Alton et al. (WO 83/04053), one can readily design and manufacture genes encoding microbial expression of SCF polypeptides having primary conformations that differ from SCF in terms of the identity or location of one or more residues (e.g., substitutions, terminal and intermediate additions and deletions). Alternatively, modifications of cDNA and genomic genes can be readily accomplished by well-known site-directed mutagenesis techniques and employed to generate analogs and derivatives of SCF. Such products share at least one of the biological properties of SCF but may differ in others.

SCF polypeptides useful according to the subject invention include those that have been shortened by e.g., deletions, those that are more resistant to hydrolysis (and, therefore, may have more pronounced or longer-lasting effects than naturally-occurring SCF), those that have been altered to delete or to add one or more potential sites for O-glycosylation and/or N-glycosylation, those that have one or more cysteine residues deleted or replaced by, e.g., alanine or serine residues and are potentially more easily isolated in active form from microbial systems, and those that have one or more tyrosine residues replaced by phenylalanine and bind to a target receptor. SCF analogs with increased biological activity and stability could be desirable as lower dosages may be used to achieve the same biological result. Also contemplated are polypeptide fragments having only a part of the continuous amino acid sequence or secondary conformations within SCF, which fragments may possess one property of SCF (e.g., receptor binding) and not others (e.g., early hematopoietic cell growth activity).

Thus, the SCF can be the full-length native polypeptide or a variant of the sequence. See e.g., Martin, F H. Cell, 1990, 63, 203-211; U.S. Pat. No. 6,218,148. The SCF polypeptide can also be a truncated or secreted form of an SCF polypeptide, (e.g., soluble forms containing an extracellular domain sequence), variant forms (e.g., alternatively spliced forms) and allelic variants of an SCF polypeptide.

A full length native SCF polypeptide is produced as a 273 amino acid precursor, which comprises residues 1-25 as the signal sequence, residues 26-214 as the extracellular domain, residues 215-237 as a potential transmembrane domain and residues 238-273 as a potential cytoplasmic domain. See Swiss-Prot entry P21583 or FIG. 12. A nucleotide sequence encoding a full-length native human SCF is shown in FIG. 13 as nt positions 41-862 (see also GenBank accession no. BC074725). Nucleic acid sequences encoding splice variants of SCF are found in GenBank under accession no. NM.sub.—000899 and NM.sub.—003994.2. Thus, a human native SCF represents 248 amino acids from positions 26-273 of Swiss-Prot entry P21583 or FIG. 12. Shorter versions of the native human SCF include a membrane bound form representing positions 26-245 of Swiss-Prot entry P21583 or FIG. 12 and a soluble version representing positions 26-165 of Swiss-Prot entry P21583 or FIG. 12.

Fragments, analogs, variants, and derivatives of SCF are reported in, for example, U.S. Pat. Nos. 6,204,363; 6,207,417; 6,207,454; 6,207,802; 6,218,148; and 6,248,319.

As used herein, “soluble SCF” or “secretable SCF” refers to a form of SCF that has biological activity with respect to the c-kit but which lacks a functional transmembrane domain. Secretable forms of SCF that lack a functional transmembrane domain are missing all or nearly all of the sequence from 238-273 of Swiss-Prot entry P21583 or FIG. 12.

SCF polypeptides also may include pre- or pro-proteins or mature proteins, including polypeptides or proteins that are capable of being directed to the endoplasmic reticulum (ER), a secretory vesicle, a cellular compartment, or an extracellular space typically, e.g., as a result of a signal sequence; however, proteins released into an extracellular space without necessarily having a signal sequence are also encompassed. Generally, the polypeptides undergo processing, e.g., cleavage of a signal sequence, modification, folding, etc., resulting in a mature form (see, e.g., Alberts, et al. (1994) Molecular Biology of The Cell, Garland Publishing, New York, N.Y., pp. 557-592). If an SCF polypeptide is released into the extracellular space, it can undergo extracellular processing to produce a “mature” protein. Release into the extracellular space can occur by many mechanisms, including, e.g., exocytosis, and proteolytic cleavage.

The term “mature protein” or “mature polypeptide” as used herein refers to the form(s) of the protein produced by expression in a mammalian cell. It is generally hypothesized that once export of a growing protein chain across the rough endoplasmic reticulum has been initiated, proteins secreted by mammalian cells have a signal peptide (SP) sequence that is cleaved from the complete polypeptide to produce a “mature” form of the protein. Cleavage of a secreted protein is not often uniform and may result in more than one species of mature protein. The cleavage site of a secreted protein is determined from the primary amino acid sequence of the complete protein.

SCF polypeptides may also be “altered,” resulting in “variations,” and may contain deletions, insertions, or substitutions of amino acid residues that produce a silent change and result in functionally equivalent proteins. Deliberate amino acid substitutions may be made based on similarity in polarity, charge, solubility, hydrophobicity, hydrophilicity, and/or the amphipathic nature of the residues, as long as the relevant biological or immunological activity of the SCF polypeptide is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

“Variant SCF polypeptides” refer to an “active” SCF polypeptide, wherein activity is as defined herein, and having at least about 80% amino acid sequence identity with the native human SCF polypeptide sequence. Such SCF polypeptide variants include, for instance, SCF polypeptides wherein one or more amino acid residues are added, substituted or deleted at the N- or C-terminus or within the sequence. Ordinarily, variant SCF polypeptides will have at least about 90% amino acid sequence identity, more preferably at least about 95% sequence identity, 96%, 97%, 98%, 99% or greater than 99% sequence identity with the native amino acid sequence described, with or without the signal peptide. Natural and nonnatural SCF polypeptides are also described in, for example, U.S. Pat. No. 6,759,215 or 6,207,417.

The term “identity” describes amino acid residues, which are identical between different amino acid sequences. “Percent (%) amino acid sequence identity” with respect to the SCF amino acid sequences identified herein is defined as the percentage of amino acid residues in a candidate sequence that are identical with the amino acid residues in an SCF polypeptide sequence, after aligning the sequences and introducing gaps, if necessary, to achieve the maximum percent sequence identity, and not considering any conservative substitutions as part of the sequence identity.

Derivatives of SCF are also contemplated herein. Such derivatives include molecules modified by one or more water soluble polymer molecules, such as polyethylene glycol, or by the addition of polyamino acids, including fusion proteins (procedures for which are well-known in the art). Such derivatization may occur singularly at the N- or C-terminus or there may be multiple sites of derivatization. Substitution of one or more amino acids with lysine may provide additional sites for derivatization. (See U.S. Pat. No. 5,824,784 and U.S. Pat. No. 5,824,778, incorporated by reference herein).

SCF polypeptides can be prepared in any manner known in the art. For example, SCF polypeptides can be isolated, recombinantly produced, synthetically produced, or produced by any combination of these methods. For example, a recombinantly produced version of an SCF polypeptide, including a secreted polypeptide, can be purified using techniques described herein or otherwise known in the art. See Martin F H, et. al., Primary structure and functional expression of rat and human stem cell factor DNAs. Cell 63:203, 1990.

### Polynucleotides Encoding SCF

SCF polypeptides and variants described herein are encoded by nucleic acids. A SCF-encoding polynucleotide can be composed of polyribonucleotide or polydeoxyribonucleotide, which may be unmodified RNA or DNA or modified RNA or DNA. For example, the SCF polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, an encoding SCF polynucleotide can be composed of triple-stranded regions comprising RNA or DNA or both RNA and DNA. SCF polynucleotides may also contain one or more modified bases or DNA or RNA backbones modified for stability or for other reasons. “Modified” bases include, for example, tritulated bases and unusual bases such as inosine. A variety of modifications can be made to DNA and RNA; thus, “polynucleotide” embraces chemically, enzymatically, or metabolically modified forms.

An SCF polynucleotide variant will typically have at least about 70% nucleic acid sequence identity, more preferably at least about 80% nucleic acid sequence identity, yet more preferably at least about 85% nucleic acid sequence identity, yet more preferably at least about 90% nucleic acid sequence identity, yet more preferably at least about 95% nucleic acid sequence identity with a native SCF nucleic acid sequence. Preferably, the variants will have at least about 95%, more preferably at least 96%, 97%, 98%, 99% or greater than 99% sequence identity to the native polynucleotide sequence. See Martin, F H. Primary Structure and Functional Expression of Rat And Human Stem-Cell Factor DNAS Cell, 1990, 63, 203-211.

It will be appreciated by those skilled in the art that as a result of the degeneracy of the genetic code, a multitude of polynucleotide sequences encoding SCF polypeptides, some bearing minimal homology to the polynucleotide sequences of any known and naturally occurring gene, may be produced. Thus, the SCF encoding nucleic acid contemplates each and every possible variation of polynucleotide sequence that could be made by selecting combinations based on possible codon choices. These combinations are made in accordance with the standard triplet genetic code as applied to the polynucleotide sequences of naturally occurring SCF, and all such variations are to be considered as being specifically disclosed. DNA sequences encoding natural and nonnatural SCF polypeptides are described in U.S. Pat. No. 6,759,215 or 6,207,417.

“Percent (%) nucleic acid sequence identity” with respect to the SCF polynucleotide sequences identified herein is defined as the percentage of nucleotides in a candidate sequence that are identical with the nucleotides in the SCF polynucleotide sequence after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software and using default settings for gap penalty and the like. For purposes herein, a percent amino acid sequence identity value is determined by dividing (a) the number of matching identical amino acid residues between the amino acid sequence of the SCF polypeptide of interest and the comparison amino acid sequence of interest (i.e., the sequence against which the SCF polypeptide of interest is being compared) as determined by WU-BLAST-2, by (b) the total number of amino acid residues of the SCF polypeptide of interest, respectively.

In other embodiments, the SCF polypeptides are encoded by nucleic acid molecules which are capable of hybridizing, preferably under stringent hybridization and wash conditions, to nucleotide sequences encoding the full-length SCF native polypeptide. Exemplary high stringency conditions include low salt concentrations (e.g., <4.times.SSC buffer and/or <2.times.SSC buffer), the presence of non-ionic detergent (e.g., 0.1% SDS), and/or relatively high temperatures (e.g., >55.degree. C. and/or >70.degree. C.).

The polynucleotide may be prepared by synthetic chemistry. After production, the synthetic sequence may be used alone or inserted into any of the many available expression vectors and cell systems using reagents that are well known in the art. Moreover, synthetic chemistry may be used to introduce mutations into a sequence encoding SCF.

### Vectors

As used herein, the term “vector” means a non-chromosomal nucleic acid comprising an intact replicon such that the vector may be replicated when placed within a cell, for example, by a process of transformation. Viral vectors include retroviruses, adenoviruses, herpesvirus, papovirus, or otherwise modified naturally occurring viruses. Vector also means a formulation of nucleic acid with a chemical or substance which allows uptake by cells.

Vectors for delivering nucleic acids can be viral, non-viral, or physical. See, for example, Rosenberg et al., Science, 242:1575-1578 (1988), and Wolff et al., Proc. Natl. Acad. Sci. USA 86:9011-9014 (1989). Reviews discussing methods and compositions for use in gene therapy include Eck et al., in Goodman & Gilman's The Pharmacological Basis of Therapeutics, Ninth Edition, Hardman et al., eds., McGray-Hill, New York, (1996), Chapter 5, pp. 77-101; Wilson, Clin. Exp. Immunol. 107 (Suppl. 1):31-32 (1997); Wivel et al., Hematology/Oncology Clinics of North America, Gene Therapy, S. L. Eck, ed., 12(3):483-501 (1998); Romano et al., Stem Cells, 18:19-39 (2000), and the references cited therein. U.S. Pat. No. 6,080,728 also provides a discussion of a wide variety of gene delivery methods and compositions. The routes of delivery include, for example, systemic administration and administration in situ. Well-known viral delivery techniques include the use of adenovirus, retrovirus, lentivirus, foamy virus, herpes simplex virus, and adeno-associated virus vectors.

Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA. In vivo DNA-mediated gene transfer into a variety of different target sites has been studied extensively. DNA for gene transfer also may be used in association with various cationic lipids, polycations and other conjugating substances. See Przybylska et al., J. Gene Med., 2004 6: 85-92; Svahn, et al., J. Gene Med., 2004 6: S36-S44.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences are functionally linked and in some cases contiguous such as a secretory leader, which is contiguous also in reading frame. However enhancers do not have to be contiguous. Linking is accomplished by ligation at convenient restriction sites. If such sites do not exist, the synthetic oligonucleotide adapters or linkers are used in accordance with conventional practice.

The techniques for introducing nucleic acids into cells vary depending upon whether the nucleic acid is transferred into cultured cells in vitro or in vivo into cells of the intended host. Techniques suitable for the transfer of nucleic acid into mammalian cells in vitro include the use of liposomes, electroporation, microinjection, cell fusion, DEAE-dextran, the calcium phosphate precipitation method, and the like. Preferred in vivo gene transfer techniques include transfection with viral (typically, retroviral or adenoviral) vectors and viral coat protein-liposome mediated transfection (Dzau, et al., Trends in Biotechnology 11 (5):205-10 (1993)). Suitable vectors can be constructed by any of the methods well known in the art. See, for example, Sambrook et al., Molecular Cloning, A Laboratory Manual, Second Edition, Cold Spring Harbor Press (1989), and Ausubel et al., eds., Current Protocols in Molecular Biology, John Wiley & Sons, N.Y. (1987 and updates); Moore, et al., Ann N Y Acad. Sci. 2001 938:36-45-47. The use of cationic liposomes, such as the CD-Chol/DOPE liposome, has been widely documented as an appropriate vehicle to deliver DNA to a wide range of tissues through intravenous injection of DNA/cationic liposome complexes. See Caplen et al., Nature Med., 1:39-46 (1995). Liposome transfer of genes to target cells by fusing with the plasma membrane. Examples of the successful application of liposome complexes include those of Lesson-Wood et al., Human Gene Therapy, 6:395-405 (1995), and Xu et al., Molecular Genetics and Metabolism, 63:103-109 (1998).

### Pharmaceutical Compositions

SCF can be incorporated into pharmaceutical compositions. Such compositions typically include SCF and a pharmaceutically acceptable carrier.

Aqueous compositions of the present invention comprise an effective amount of SCF, an expression vector, or cells, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well-known in the art. Supplementary active compounds can also be incorporated into the compositions.

Such compositions can include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., thimersol, benzyl alcohol), and bulking substances (e.g., lactose, mannitol); incorporation of the material into particulate preparations of polymeric compounds, such as polylactic acid, polyglycolic acid, etc., or in association with liposomes or micelles. Such compositions will influence the physical state, stability, rate of in vivo release, and rate of in vivo clearance of SCF. See, e.g., Remington's Pharmaceutical Sciences, 18th Ed. (1990) Mack Publishing Co., Easton, Pa., pages 1435-1712, which are herein incorporated by reference.

SCF or derivatives thereof may be formulated for injection, or oral, nasal, pulmonary, topical, or other types of administration as one skilled in the art will recognize. The formulation may be liquid or may be solid, such as lyophilized, for reconstitution.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. A parenteral preparation can be enclosed in ampoules, disposable syringes or multiple dose vials made of glass or plastic.

Pharmaceutical compositions suitable for injectable use include sterile aqueous solutions or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition should be sterile and fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prolonged absorption of the injectable compositions can be brought about by including in the composition an agent that delays absorption, for example, aluminum monostearate and gelatin.

Sterile injectable solutions can be prepared by incorporating the active compound(s) in the required amount in an appropriate solvent with one or a combination of ingredients enumerated, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation can include vacuum drying or freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid.

Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The compositions used in the methods of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. Dosage units can also be accompanied by instructions for use.

“Unit dose” is defined as a discrete amount of a therapeutic composition dispersed in a suitable carrier. For example, where polypeptides are being administered parenterally, the polypeptide compositions are generally injected in doses ranging from 1 μg/kg to 100 mg/kg body weight/day, preferably at doses ranging from 0.1 mg/kg to about 50 mg/kg body weight/day. Parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The pharmaceutical compositions can be included in a container, pack, or dispenser together with instructions for administration.

In addition, the present invention contemplates a kit containing SCF, and optionally, at least one additional factor.

### Methods of Administration

In accordance with the subject invention, SCF can be used to treat a subject that is at risk for, or has, a glucose transport-related disorder such as type II diabetes. Methods of identifying such individuals are known in the art. Thus, the subject invention pertains to methods and compositions for both prophylactic and therapeutic methods of treating a subject at risk of (or susceptible to) a disorder or having a disorder associated with aberrant glucose transport.

The present invention contemplates methods using pharmaceutical compositions comprising effective amounts of SCF polypeptide, with pharmaceutically acceptable diluents, preservatives, solubilizers, emulsifiers, adjuvants and/or carriers useful in SCF therapy.

Administration of these compositions according to the present invention will be via any common route so long as the target tissue is available via that route. The pharmaceutical compositions may be introduced into the subject by any conventional method, e.g., by intravenous, intradermal, intramuscular, intramammary, intraperitoneal, intrathecal, retrobulbar, intrapulmonary, oral, sublingual, nasal, anal, vaginal, or transdermal delivery, or by surgical implantation at a particular site. The treatment may consist of a single dose or a plurality of doses over a period of time.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

Generally, effective amounts of SCF, or a derivative thereof, will be determined by the age, weight, and condition or severity of disease of the recipient. See, Remington's Pharmaceutical Sciences, supra, pages 697-773, herein incorporated by reference. Typically, a dosage of between about 0.001 μg/kg body weight/day to about 1000 μg/kg body weight/day, may be used, but more or less, as a skilled practitioner will recognize, may be used. Dosing may be one or more times daily, or less frequently, and may be in combination with other compositions as described herein. It should be noted that the present invention is not limited to the dosages recited herein.

As defined herein, a therapeutically effective amount of protein will generally range from about 0.001 to 30 mg/kg body weight, about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, or 5 to 6 mg/kg body weight. The protein can be administered one time per week for between about 1 to 10 weeks, generally between 2 to 8 weeks, between about 3 to 7 weeks, or for about 4, 5, or 6 weeks. One in the art will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein can include a single treatment or can include a series of treatments. In the case of a subject suffering from diabetes, blood glucose levels can be monitored and the dosages adjusted accordingly.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, supra, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The final dosage regimen will be determined by the attending physician, considering factors that modify the action of drugs, e.g., the drug's specific activity, severity of the damage and the responsiveness of the patient, the age, condition, body weight, sex and diet of the patient, the severity of any infection, time of administration and other clinical factors.

It will be appreciated that the pharmaceutical compositions and treatment methods of the invention may be useful in fields of human medicine and veterinary medicine. Thus the subject to be treated may be a mammal, preferably a human.

### Combination Therapy

In accordance with the present invention, SCF will, in some instances, be used in combination with other therapeutic agents to bring about a desired effect. Selection of additional agents will, in large part, depend on the desired target therapy. Combination therapy includes administration of a single pharmaceutical dosage formulation that contains SCF and one or more additional active agents, as well as administration of SCF and each active agent in its own separate pharmaceutical dosage formulation. For example, SCF and a DPP-IV inhibitor can be administered to the human subject together in a single composition, or each agent can be administered in separate formulations. Where separate formulations are used, SCF and one or more additional active agents can be administered at essentially the same time (i.e., concurrently), or at separately staggered times (i.e., sequentially).

An example of combination therapy can be seen in modulating (preventing the onset of the symptoms or complications associated with) diabetes (or treating, preventing or reducing the risk of developing, diabetes and its related symptoms, complications, and disorders), wherein SCF can be effectively used in combination with, for example, biguanides (such as metformin); thiazolidinediones (such as ciglitazone, pioglitazone, troglitazone, and rosiglitazone); dipeptidyl-peptidase-4 (“DPP-IV”) inhibitors (such as vildagliptin and sitagliptin); glucagonlike peptide-1 (“GLP-1”) receptor agonists (such as exanatide) (or GLP-1 mimetics); PPAR gamma agonists or partial agonists; dual PPAR alpha, PPAR gamma agonists or partial agonists; dual PPAR delta, PPAR gamma agonists or partial agonists; pan PPAR agonists or partial agonists; dehydroepiandrosterone (also referred to as DHEA or its conjugated sulphate ester, DHEA-SO.sub.4); antiglucocorticoids; TNF.alpha. inhibitors; .alpha.-glucosidase inhibitors (such as acarbose, miglitol, and voglibose); sulfonylureas (such as chlorpropamide, tolbutamide, acetohexamide, tolazamide, glyburide, gliclazide, glynase, glimepiride, and glipizide); pramlintide (a synthetic analog of the human hormone amylin); other insulin secretogogues (such as repaglinide, gliquidone, and nateglinide); insulin (or insulin mimetics); glucagon receptor antagonists; gastric inhibitory peptide (“GIP”); or GIP mimetics; as well as the active agents discussed below for treating obesity, hyperlipidemia, atherosclerosis and/or metabolic syndrome.

Another example of combination therapy can be seen in treating obesity or obesity-related disorders, wherein SCF can be effectively used in combination with, for example, phenylpropanolamine, phenteramine; diethylpropion; mazindol; fenfluramine; dexfenfluramine; phentiramine, .beta.-3 adrenoceptor agonist agents; sibutramine; gastrointestinal lipase inhibitors (such as orlistat); and leptins. Other agents used in treating obesity or obesity-related disorders wherein the compounds of Formula (I) can be effectively used in combination with, for example, cannabinoid-1 (“CB-1”) receptor antagonists (such as rimonabant); PPAR delta agonists or partial agonists; dual PPAR alpha, PPAR delta agonists or partial agonists; dual PPAR delta, PPAR gamma agonists or partial agonists; pan PPAR agonists or partial agonists; neuropeptide Y; enterostatin; cholecytokinin; bombesin; amylin; histamine H.sub.3 receptors; dopamine D.sub.2 receptors; melanocyte stimulating hormone; corticotrophin releasing factor; galanin; and gamma amino butyric acid (GABA).

Still another example of combination therapy can be seen in modulating hyperlipidemia (treating hyperlipidemia and its related complications), wherein SCF can be effectively used in combination with, for example, statins (such as atorvastatin, fluvastatin, lovastatin, pravastatin, and simvastatin), CETP inhibitors (such as torcetrapib); a cholesterol absorption inhibitor (such as ezetimibe); PPAR alpha agonists or partial agonists; PPAR delta agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; fenofibric acid derivatives (such as gemfibrozil, clofibrate, fenofibrate, and bezafibrate); bile acid-binding resins (such as colestipol or cholestyramine); nicotinic acid; probucol; betacarotene; vitamin E; or vitamin C.

A further example of combination therapy can be seen in modulating atherosclerosis, wherein SCF is administered in combination with one or more of the following active agents: an antihyperlipidemic agent; a plasma HDL-raising agent; an antihypercholesterolemic agent, such as a cholesterol biosynthesis inhibitor, e.g., an hydroxymethylglutaryl (HMG) CoA reductase inhibitor (also referred to as statins, such as lovastatin, simvastatin, pravastatin, fluvastatin, and atorvastatin); an HMG-CoA synthase inhibitor; a squalene epoxidase inhibitor; or a squalene synthetase inhibitor (also known as squalene synthase inhibitor); an acyl-coenzyme A cholesterol acyltransferase (ACAT) inhibitor, such as melinamide; probucol; nicotinic acid and the salts thereof and niacinamide; a cholesterol absorption inhibitor, such as .beta.-sitosterol; a bile acid sequestrant anion exchange resin, such as cholestyramine, colestipol or dialkylaminoalkyl derivatives of a cross-linked dextran; an LDL receptor inducer; fibrates, such as clofibrate, bezafibrate, fenofibrate, and gemfibrizol; vitamin B.sub.6 (also known as pyridoxine) and the pharmaceutically acceptable salts thereof, such as the HCl salt; vitamin B.sub.12 (also known as cyanocobalamin); vitamin B.sub.3 (also known as nicotinic acid and niacinamide); anti-oxidant vitamins, such as vitamin C and E and beta carotene; a beta-blocker, an angiotensin II antagonist; an angiotensin converting enzyme inhibitor; PPAR alpha agonists or partial agonists; PPAR delta agonists or partial agonists; PPAR gamma agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; and a platelet aggregation inhibitor, such as fibrinogen receptor antagonists (i.e., glycoprotein IIb/IIIa fibrinogen receptor antagonists) and aspirin. As noted above, the compounds of Formula (I) can be administered in combination with more than one additional active agent, for example, a combination of a compound of Formula (I) with an HMG-CoA reductase inhibitor (e.g., atorvastatin, fluvastatin, lovastatin, pravastatin, and simvastatin) and aspirin, or a compound of Formula (I) with an HMG-CoA reductase inhibitor and a .beta. blocker.

Additionally, an effective amount of SCF and a therapeutically effective amount of one or more active agents selected from the group consisting of: an antihyperlipidemic agent; a plasma HDL-raising agent; an antihypercholesterolemic agent, such as a cholesterol biosynthesis inhibitor, for example, an HMG-CoA reductase inhibitor; an HMG-CoA synthase inhibitor; a squalene epoxidase inhibitor, or a squalene synthetase inhibitor (also known as squalene synthase inhibitor); an acyl-coenzyme A cholesterol acyltransferase inhibitor; probucol; nicotinic acid and the salts thereof; CETP inhibitors such as torcetrapib; a cholesterol absorption inhibitor such as ezetimibe; PPAR alpha agonists or partial agonists; PPAR delta agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; niacinamide; a cholesterol absorption inhibitor; a bile acid sequestrant anion exchange resin; a LDL receptor inducer; clofibrate, fenofibrate, and gemfibrozil; vitamin B.sub.6 and the pharmaceutically acceptable salts thereof; vitamin B.sub.12; an anti-oxidant vitamin; a .beta.-blocker; an angiotensin II antagonist; an angiotensin converting enzyme inhibitor; a platelet aggregation inhibitor; a fibrinogen receptor antagonist; aspirin; phentiramines, .beta.-3 adrenergic receptor agonists; sulfonylureas, biguanides, .alpha.-glucosidase inhibitors, other insulin secretogogues, and insulin can be used together for the preparation of a pharmaceutical composition useful for the above-described treatments.

An additional example of combination therapy can be seen in modulating metabolic syndrome (or treating metabolic syndrome and its related symptoms, complications and disorders), wherein SCF can be effectively used in combination with, for example, the active agents discussed above for modulating or treating diabetes, obesity, hyperlipidemia, atherosclerosis, and/or their respective related symptoms, complications and disorders.

In a further embodiment, a compound of the present invention can be administered in combination with halofenic acid, an ester of halofenic acid, or another prodrug of halofenic acid, preferably with (−)-(4-chlorophenyl)-(3-trifluoromethylphenoxy)-acetic acid 2-acetylaminoethyl ester (MBX-102).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### Materials and Methods

### Human Subjects and Experimental Animals

After obtaining informed consent, samples of subcutaneous abdominal fat and rectus abdominis muscle of human subjects were collected. Mice heterozygous for c-kit mutation (KitW/Kit+ and KitWν/Kit+), those with c-kit double heterozygosity (KitW/KitWν), and congenic wild-type mice (Kit+/Kit+) were obtained from the Jackson Laboratory (Bar Harbor, Me.) and bred in-house. C57BL/6 mice were obtained from Taconic (Hudson, N.Y.). Mice were fed a standard laboratory chow (Rodent Diet 5010, LabDiet, St. Louis, Mo.). Mice received humane care in accordance with the ‘Principles of laboratory animal care’ promulgated by the National Institutes of Health.

### Glucose, Insulin and Cholesterol Assays

Following overnight fasting for 15 hours, unanesthetized mice were weighed (Scout Pro balance SP202, Ohaus, Pine Brook, N.J.) and bled from the tail. Blood glucose concentration was measured using a Contour glucometer (Bayer, Tarrytown, N.Y.). Serum insulin and cholesterol concentrations were determined using an ELISA kit from Crystal Chem (Downer Grover, Ill.) and a Cholesterol LiquiColor kit from Stanbio Laboratory (Boerne, Tex.), respectively. Homeostasis Model of Assessment-Insulin Resistance (HOMA-IR) was calculated using the formula: fasting glucose (mg/dl)×fasting insulin (mU/L)/405.

### Insulin Sensitivity Test and Glucose Tolerance Test

For insulin sensitivity testing, mice were fasted for 4 hours. Blood glucose concentration was measured immediately before and 15, 30, 60, and 90 minutes after an intraperitoneal injection of regular insulin (Lilly, Indianapolis, Ind.) at 1 IU/kg body weight. For glucose tolerance testing, mice were fasted for 6 hours. Blood glucose concentration was measured immediately before and 15, 30, 60, 90, and 120 minutes after an intraperitoneal injection of dextrose at 2 g/kg body weight.

### Cell Culture and Treatments

C2C12 myoblasts were maintained in DMEM supplemented with 10% FBS at 37° C. with 5% CO2. After reaching confluence, cells were differentiated to myotubes in DMEM supplemented with 2% horse serum.

To study the effect of SCF on glucose uptake, myotubes were serum starved in DMEM supplemented with 0.1% FBS for 24 hours, then washed in PBS and incubated with 100 nM SCF, 100 nM insulin, or vehicle (DMEM) at 37° C. for 30 minutes. Glucose concentration of the medium was measured using a Contour glucometer.

To examine whether SCF stimulation resulted in activation of the insulin receptor or AMP-activated protein kinase, C2C12 myotubes were serum starved in DMEM supplemented with 0.1% FBS overnight. The following morning, C2C12 myotubes were washed in PBS and then treated with 10/20 nM SCF, 20/100 nM insulin, or vehicle (DMEM) at 37° C. for 5 minutes.

To explore the effect of SCF on GLUT4 expression, C2C12 myotubes were maintained in DMEM supplemented with 2% horse serum at 37° C. and treated with 20 nM SCF or vehicle (DMEM) for 2 days. 3T3-L1 mouse fibroblasts were maintained in DMEM supplemented with 10% normal calf serum at 37° C. with 5% CO2. After reaching confluence, 3T3-L1 cells were switched to DMEM supplemented with 10% FBS. Two days post-confluence, 3T3-L1 cells were treated with an adipogenic cocktail containing 14.5 units/dl recombinant human insulin (Lilly), 1.0 μM dexamethasone (Sigma-Aldrich), and 0.5 mM IBMX (Sigma-Aldrich). On days 4 and 6 post-confluence, cells were treated with 14.5 units/dl recombinant human insulin while still maintained in DMEM supplemented with 10% FBS.

To study the effect of SCF on glucose uptake, 3T3-L1 adipocytes were serum starved in DMEM supplemented with 0.1% FBS for 24 hours, then washed in PBS and treated with 100 nM SCF, 100 nM insulin, or vehicle (DMEM) at 37° C. for 30 minutes. Glucose concentration of the medium was measured using a Contour glucometer.

To examine whether SCF stimulation resulted in activation of the insulin receptor or AMP-activated protein kinase, adipocytes were serum starved in DMEM supplemented with 0.1% FBS for 24 hours, washed in PBS, and then treated with SCF (20/100 nM), insulin (20/100 nM), or vehicle (DMEM) at 37° C. for 5 minutes.

To explore the effect of SCF on GLUT4 expression, 3T3-L1 adipocytes were maintained in DMEM supplemented with 2% horse serum at 37° C. and treated with 20 nM SCF or vehicle (DMEM) for up to 3 days.

### SCF-Stimulated Glucose Disposal In Vivo

Recombinant mouse SCF was obtained from Invitrogen (Carlsbad, Calif.). After fasting for 5 hours, 6-week-old male C57BL/6 mice received an intraperitoneal injection of recombinant SCF (1.25 μg or 2.5 μg per mouse) or vehicle (water). Blood glucose concentration was measured 30 minutes after injection using blood sampled from the tail of unanesthetized mice and a Contour glucometer.

### Immunofluorescence Staining and Confocal Microscopy

Tissues were fixed in Carnoy's solution, embedded in paraffin, and cut into 5 μm-thick sections. Following deparaffinization, antigen retrieval was carried out in 10 mM citrate buffer, pH 6.0, heated to 95-100° C. for 20 minutes in a microwave. Sections were then blocked with 5% goat serum and sequentially incubated with: 1) c-Kit antibody (1:400; Dako, Carpinteria, Calif.), or SCF antibody (1:200; Abcam, Cambridge, Mass.), or rabbit polyclonal IgG (Isotype control for both c-Kit and SCF antibodies); 2) Tryptase antibody (1:200, Dako) or mouse IgG1 (Isotype control for tryptase antibody); 3) a 1:1 mixture of Alexa Fluor 594 goat anti-mouse and Alexa Fluor 488 goat anti-rabbit antibodies (1:1,000 each; Invitrogen); and 4) DAPI (0.1 μg/ml; Invitrogen). All incubations took place in a humid chamber at room temperature. Images were acquired on a Zeiss LSM 700 confocal microscope. Spectral emissions were selected based on chosen fluorochromes. Zeiss 40×/1.3 NA and 63×/1.4 NA Apochromat objectives were used. Images were acquired at 1024×1024 pixels with a line average of four and a pinhole at airy 1. Zeiss Zen 2009 (5.5) software was used.

### Protein Extraction and Immunoblotting

Tissue samples and cells were homogenized in ice-cold lysis buffer containing 50 mM Tris-HCl (pH 7.4), 150 mM NaCl, 1% NP-40, 0.5% sodium deoxycholate, 0.1% SDS, 5 mM EDTA, 1 mM EGTA (Boston BioProducts, Ashland, Mass.) supplemented with a cocktail of protease inhibitors (Complete Mini, Roche Applied Science, Indianapolis, Ind.). Following agitation on a rotator for 1 hour, lysates were centrifuged at 13,000 rpm for 20 minutes at 4° C. Extracted proteins were subjected to polyacrylamide gel electrophoresis using NuPAGE system (Invitrogen, Carlsbad, Calif.). Immunoblotting was carried out using antibodies from Cell Signaling (Danvers, Mass.) against: GLUT4 (1F8), insulin receptor P (4B8), insulin receptor p (Tyrl 150/1151) (19H7), insulin Receptor 3 (Tyr1146), AMPKα (23A3), phospho-AMPKα (Thr172) (40H9), AMPKβ1/2 (57C12), and phospho-AMPKβ1 (Ser108). Antibodies against SCF (Abcam, Cambridge, Mass.), c-Kit (Dako, Carpinteria, Calif.), GAPDH (6C5) (Abcam), and Actin (AC-40) (Sigma-Aldrich, St. Louis, Mo.) were also used.

### Statistics

Data are expressed as mean±SEM. Student's t-test (comparison between two groups), ANOVA with Tukey post-hoc analysis (comparison between multiple groups), and Pearson's correlation were used to analyze data (GraphPad Prism, version 5.0a). P values less than 0.05 were considered statistically significant.

### Results

SCF and c-Kit were Expressed in Skeletal Muscle and Adipose Tissue in Mice.

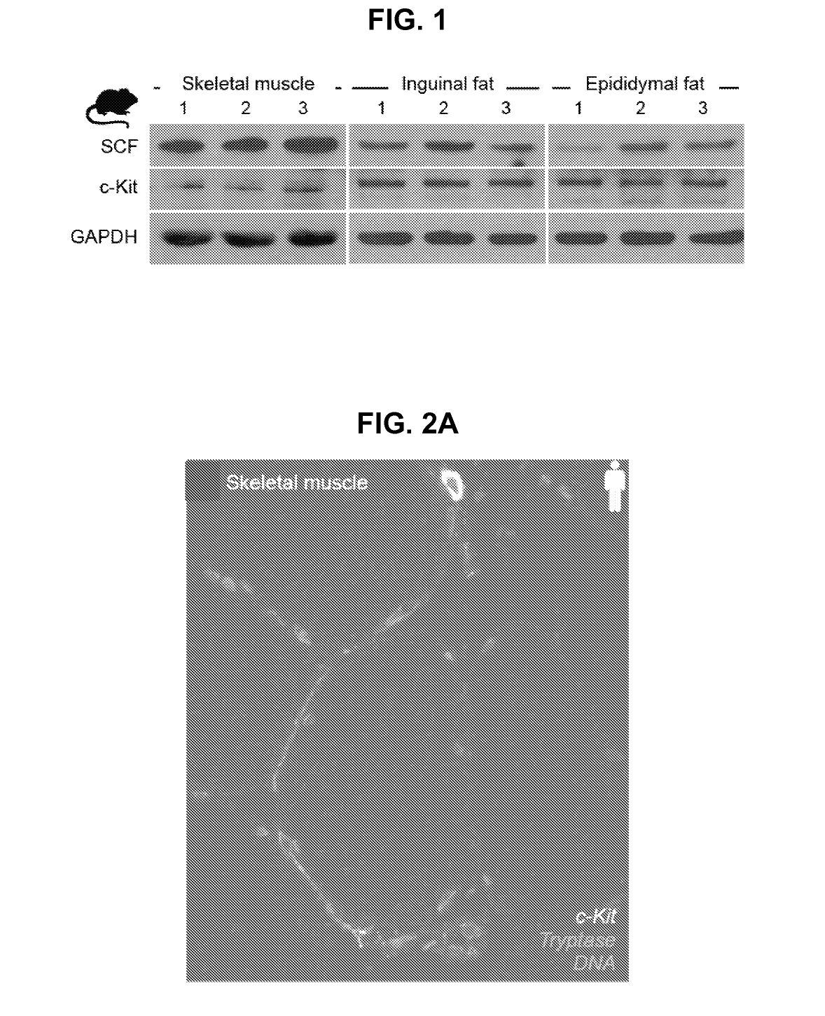

Western blot analysis of extracts from skeletal muscle, inguinal and epididymal fat of C57BL/6 mice demonstrated the expression of c-Kit and SCF in these tissues (FIG. 1).

SCF and c-Kit were Expressed in Adipose Tissue and Skeletal Muscle in Humans.

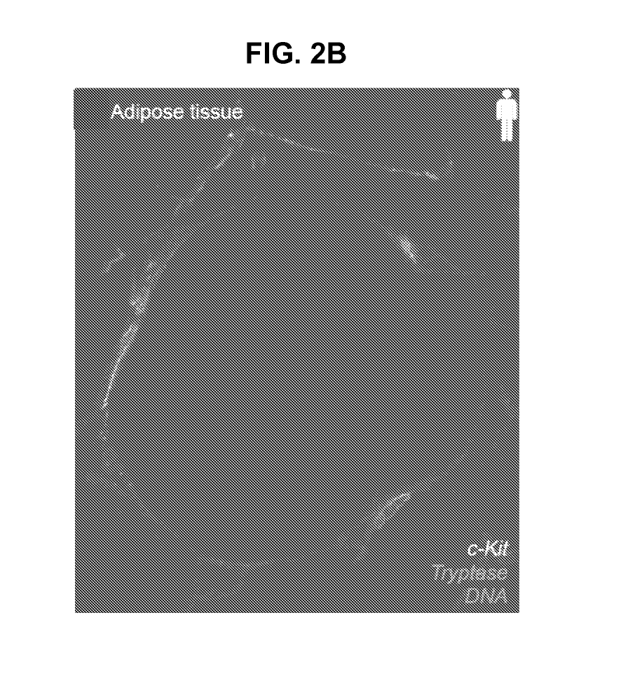

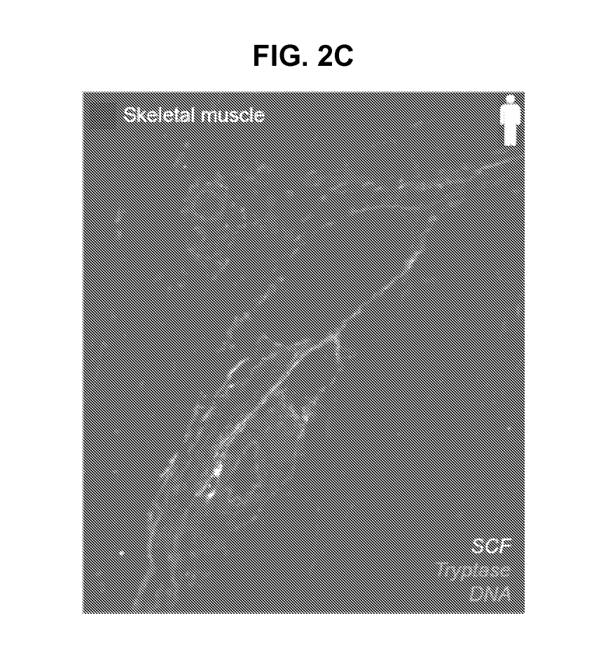

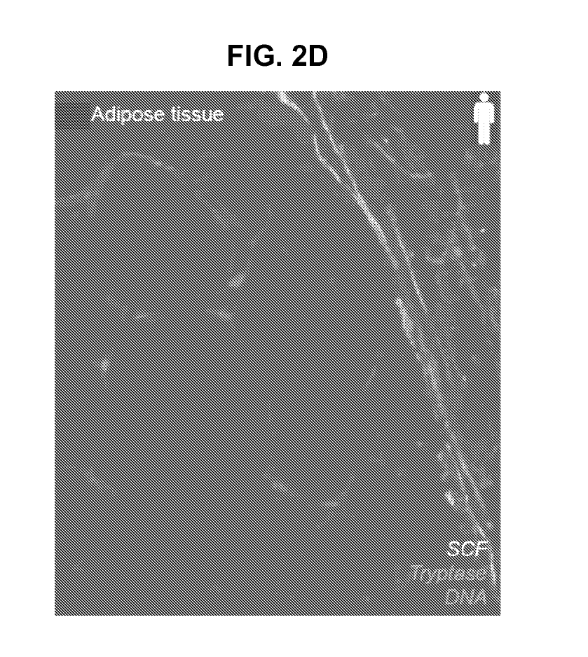

To explore whether SCF and c-Kit were also expressed in adipose tissue and skeletal muscle in human subjects, we immunostained samples of subcutaneous abdominal fat and rectus abdominis muscle (FIG. 2). Confocal microscopy revealed diffuse staining for c-Kit in both adipose tissue and skeletal muscle (FIGS. 2A,B). Since mast cells express c-Kit, we performed double immunostaining for c-Kit and tryptase, a mast cell specific marker. We found that mast cells accounted for only a fraction of c-Kit immunoreactivity in these tissues (FIG. 2A). In addition, there was a diffuse and prominent immunoreactivity for SCF in adipose tissue and skeletal muscle samples obtained from humans (FIGS. 2C,D).

SCF, c-Kit, and GLUT4 Expression Levels were Greater in Subcutaneous than in Visceral Fat in Mice.

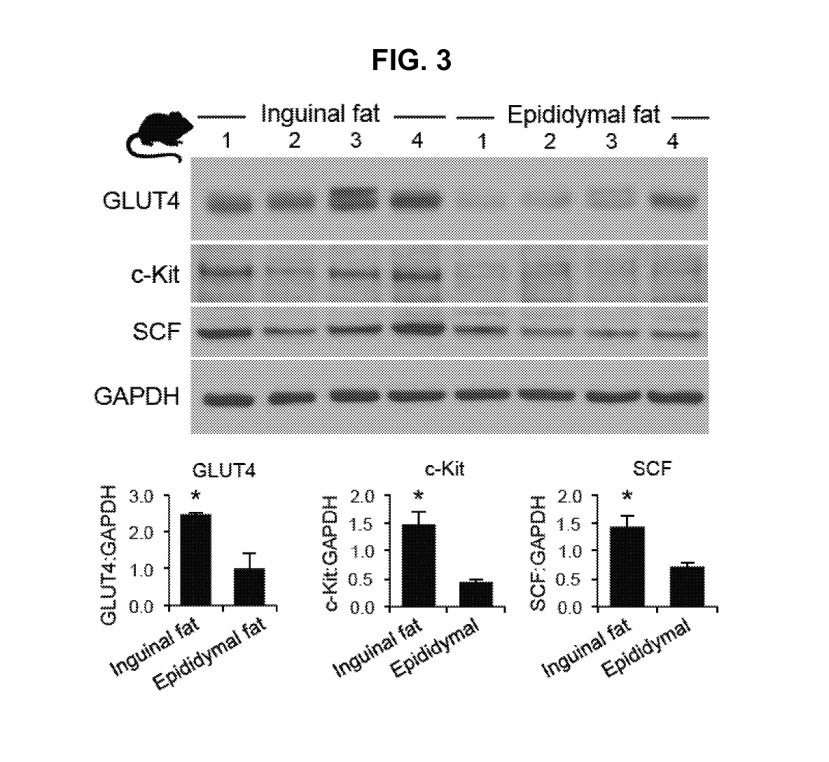

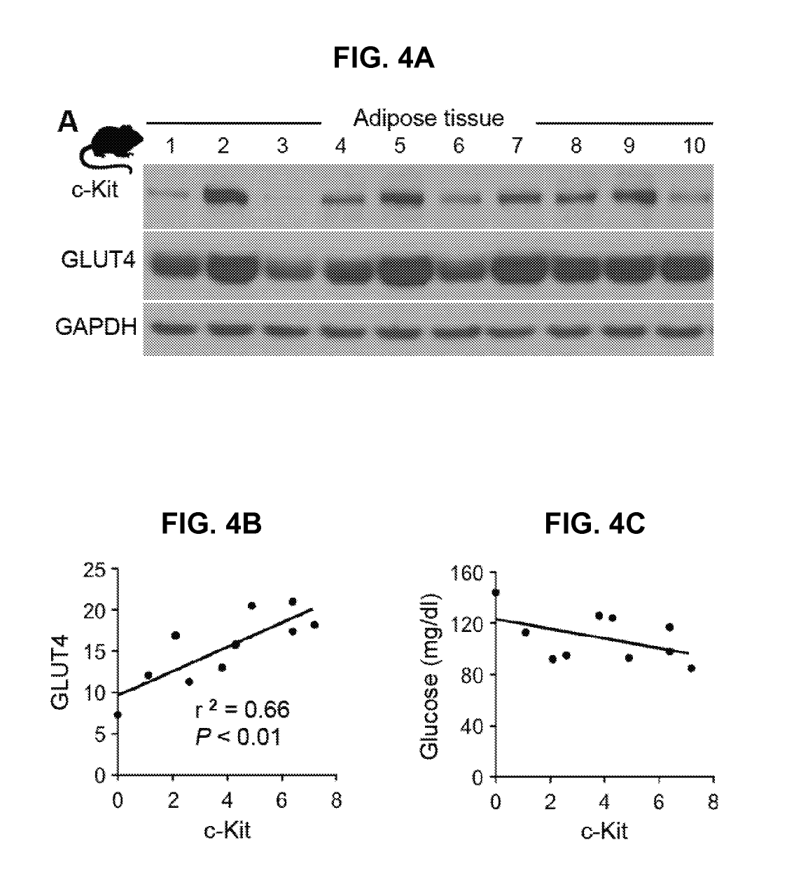

Subcutaneous fat exerts favorable metabolic effects while visceral adiposity is associated with adverse metabolic and cardiovascular consequences. We demonstrated that GLUT4 protein was expressed at significantly higher levels in inguinal (subcutaneous) than in epididymal (visceral) fat in C57BL/6 mice (FIG. 3). This was accompanied by a greater SCF and c-Kit protein expression in inguinal than in epididymal fat in C57BL/6 mice (FIG. 3). protein expression was observed in inguinal (subcutaneous) fat of C57BL/6 mice of the same age and gender (FIG. 4A). Of note, there was a direct correlation between the expression levels of c-Kit and GLUT4 (FIG. 4B). In addition, there was a trend for an inverse correlation between c-Kit expression and blood glucose concentration in these mice (FIG. 4C).

SCF Administration to Mice Resulted in an Acute and Dose-Dependent Decline in Blood Glucose Concentration.

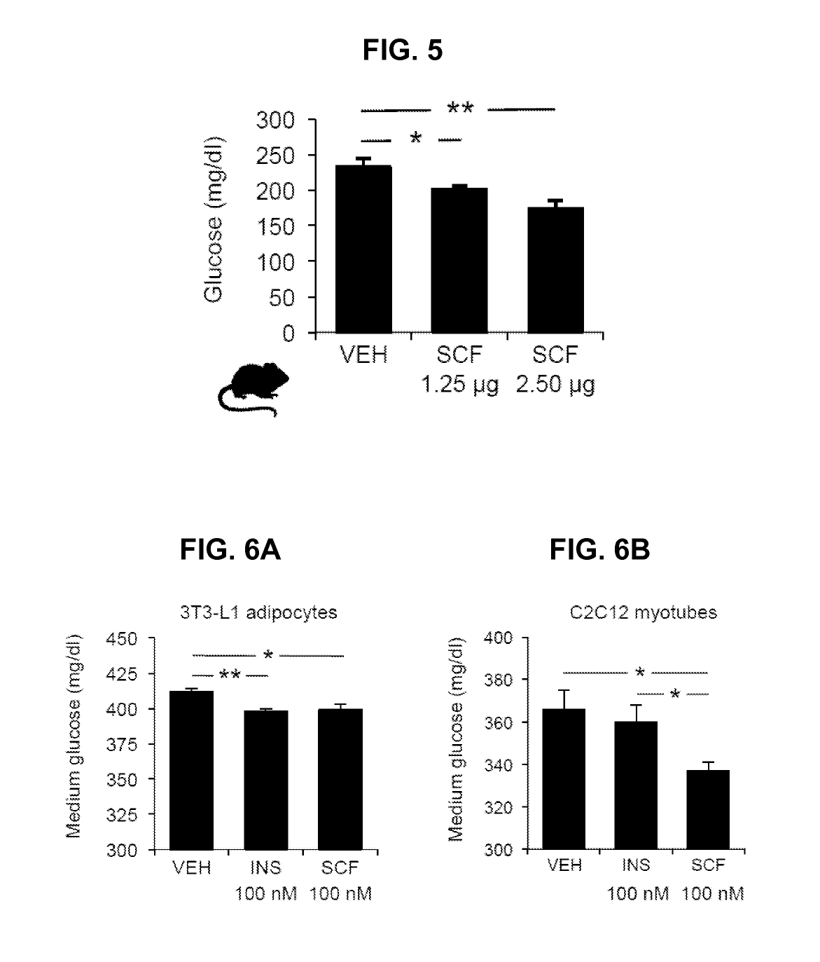

To explore the acute effect of SCF on glucose homeostasis, recombinant mouse SCF or vehicle was administered intraperitoneally to C57BL/6 mice. Thirty minutes after injection, mice treated with 1.25 μg recombinant SCF had a lower blood glucose concentration than those treated with vehicle (203±3 vs. 235±10 mg/dl, P<0.05) (FIG. 5). A further decline in blood glucose concentration was observed when mice were treated with 2.5 μg of recombinant SCF (176±9 mg/dl, P<0.01) (FIG. 5). There was no statistically significant difference in the weight of the mice between groups. Mice tolerated SCF treatment well as no apparent side effect was noted. Thus, SCF administration led to an acute and dose-dependent decline in blood glucose concentration in mice.

SCF Enhanced Glucose Uptake in Cultured Adipocytes and Myocytes Independent of the Insulin Receptor.

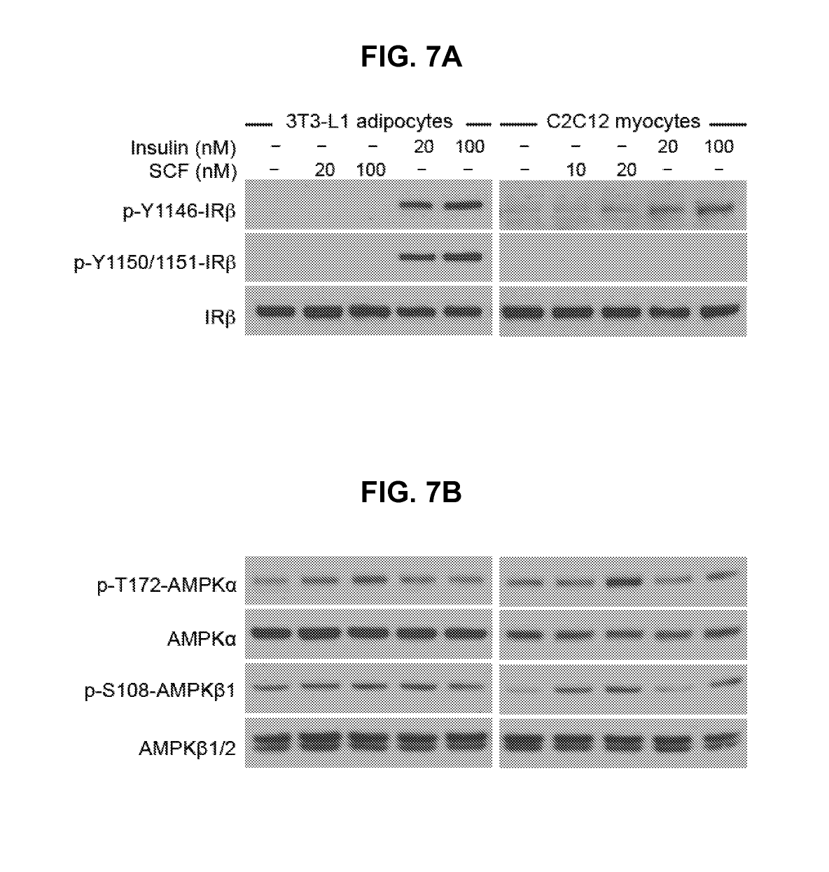

To test whether SCF also increases glucose uptake in vitro, 3T3-L1 adipocytes and C2C12 myotubes were treated with recombinant mouse SCF (FIG. 6). We demonstrated that, on an equimolar basis, SCF was as potent as insulin in promoting glucose uptake in 3T3-L1 adipocytes (FIG. 6A). In C2C12 myotubes, SCF stimulation led to an even greater increase in glucose uptake than insulin (FIG. 6B). While insulin stimulation resulted in phosphorylation of insulin receptor 13 chain at tyrosine cluster 1146/1150/1151, none of these residues were phosphorylated upon SCF stimulation of 3T3-L1 adipocytes (FIG. 7A). We also demonstrated that insulin, but not SCF, resulted in phosphorylation of tyrosine 1150/1151 of the insulin receptor 13 chain in C2C12 myotubes (FIG. 7A).

SCF Stimulation Resulted in Activating Phosphorylation of AMP-Activated Protein Kinase.

AMP-activated protein kinase (AMPK) enhances glucose uptake independent of the insulin receptor. We demonstrated that stimulation of 3T3-L1 adipocytes and C2C12 myotubes with SCF resulted in a dose-dependent phosphorylation of the a catalytic subunit of AMPK at Thr172 residue (FIG. 7B). This phosphorylation is required for AMPK activation. In addition, SCF treatment of C2C12 myotubes resulted in phosphorylation of serine 108 residue of the β1 regulatory subunit of AMPK (FIG. 7B).

SCF Treatment Increased the Expression of GLUT4 in Cultured Adipocytes and Skeletal Myocytes.

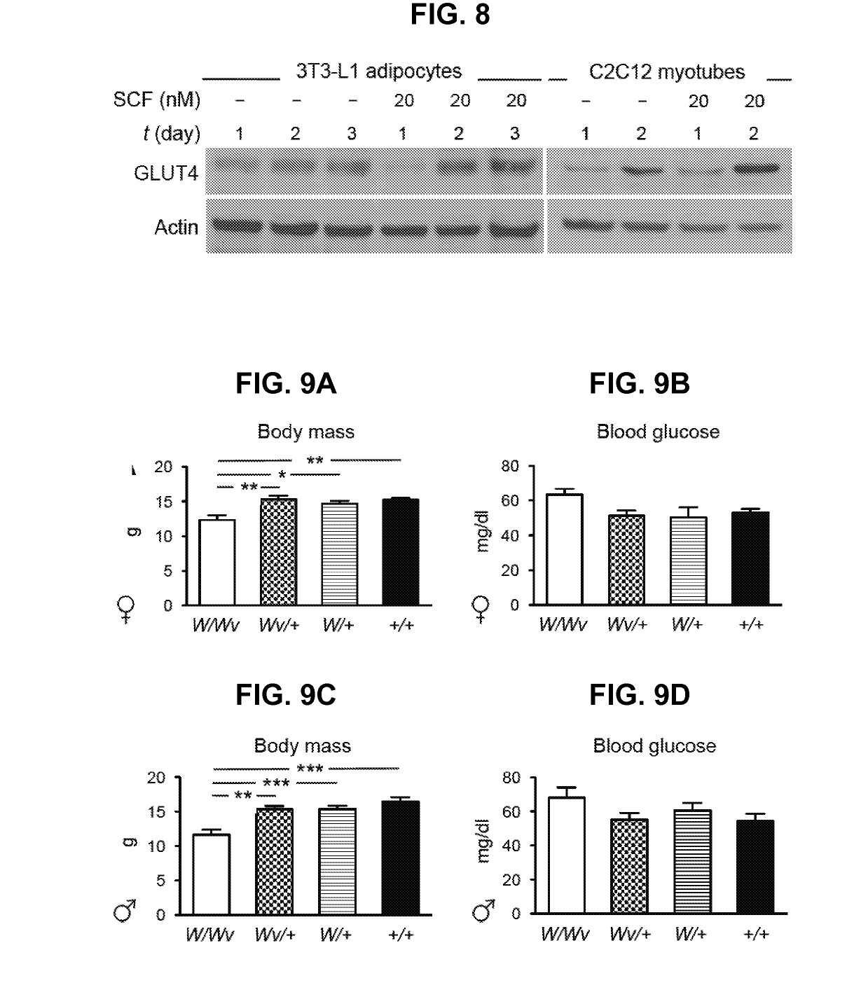

AMPK has been shown to promote GLUT4 gene expression. Therefore, we explored whether SCF stimulation of cultured adipocytes and skeletal myotubes could result in enhanced GLUT4 expression. We showed that SCF treatment resulted in increased GLUT4 protein expression in 3T3-L1 adipocytes and C2C12 myotubes (FIG. 8).

Mice with Loss-of-Function Mutations of c-Kit Showed Systemic Insulin Resistance.

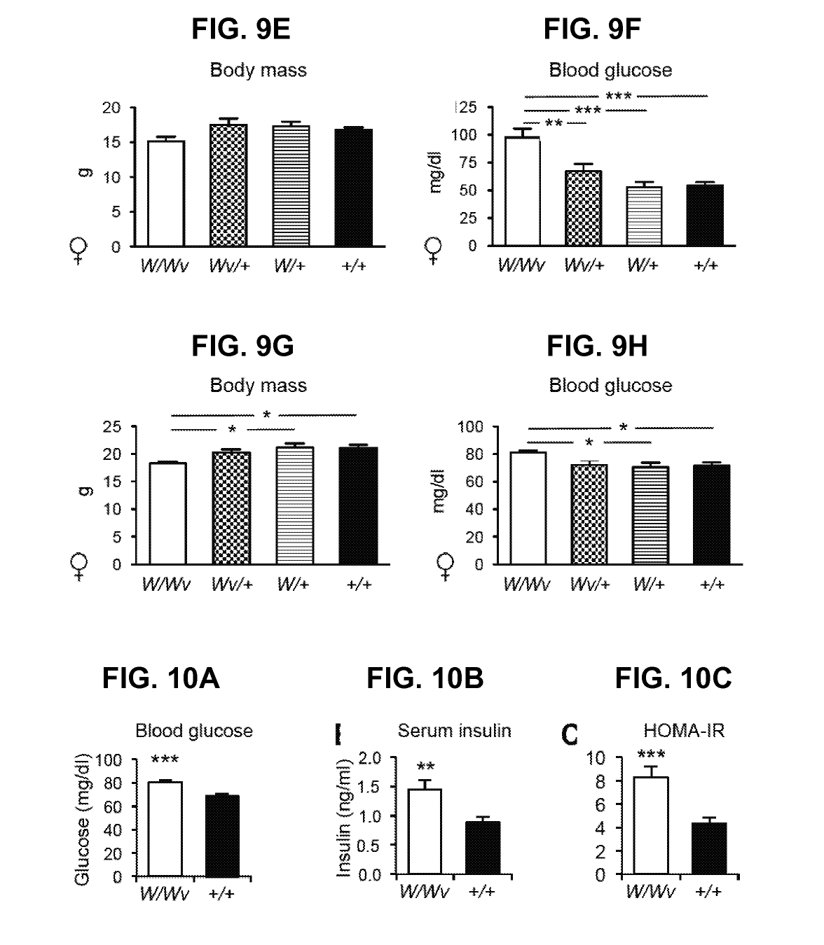

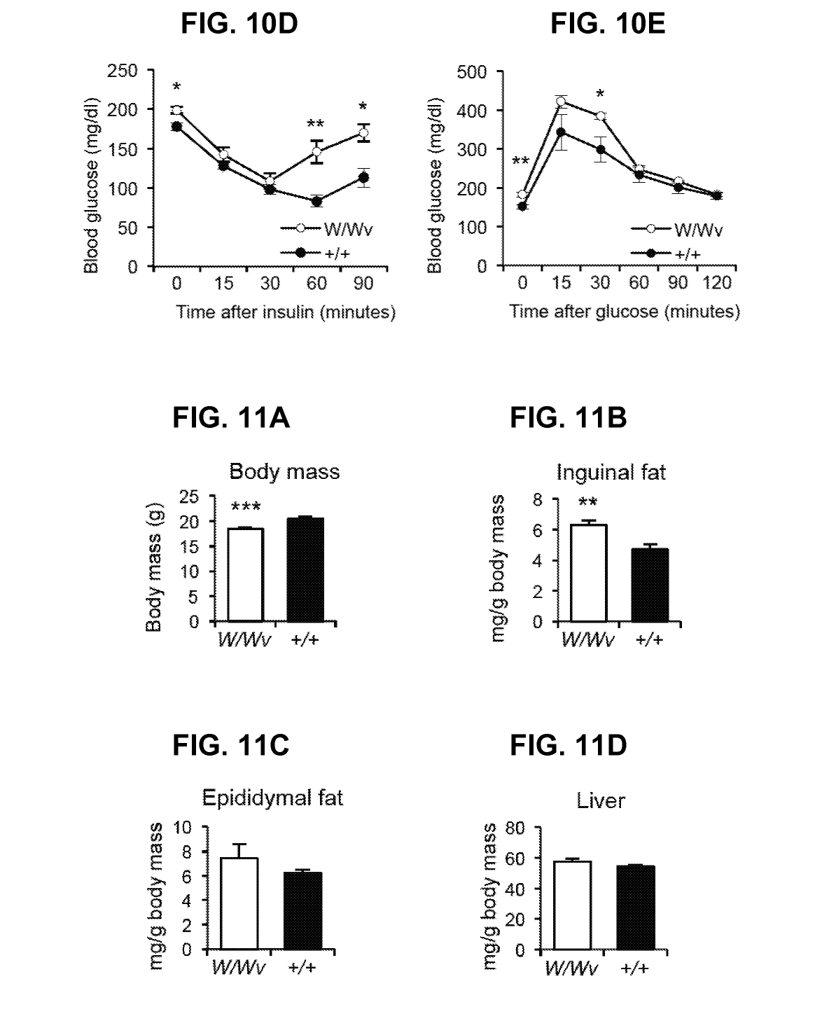

To explore the physiological role for SCF in systemic glucose homeostasis and insulin sensitivity, we examined mice with loss-of-function mutations of c-kit gene encoding the receptor for SCF. We found that mice carrying mutations involving c-kit demonstrated altered body mass and insulin sensitivity (FIGS. 9,10). Compared with KitW/Kit+, KitWν/Kit+, and wild-type littermates (Kit+/Kit+), mice with double heterozygosity for c-kit gene (KitW/KitWν) demonstrated increased fasting blood glucose concentration despite a smaller body mass (FIG. 9A-H). These changes affected both male and female mice. KitW/KitWν mice also demonstrated increased fasting glucose and serum insulin concentrations resulting in a greater insulin resistance index, as determined by the homeostasis model assessment of insulin resistance (HOMA-IR) (FIG. 10A-C). Similarly, compared with wildtype littermates, KitW/KitWν mice demonstrated attenuated glucose disposal in response to insulin (FIG. 10D). Furthermore, the glucose tolerance test revealed that KitW/KitWν mice disposed of a glucose load less efficiently than wild-type controls did (FIG. 10E). These results indicate that SCF and c-Kit interaction plays a crucial role in systemic insulin sensitivity in vivo.

c-Kit Knockout Mice Showed Diminished GLUT4 Expression in Adipose Tissue and Skeletal Muscle.

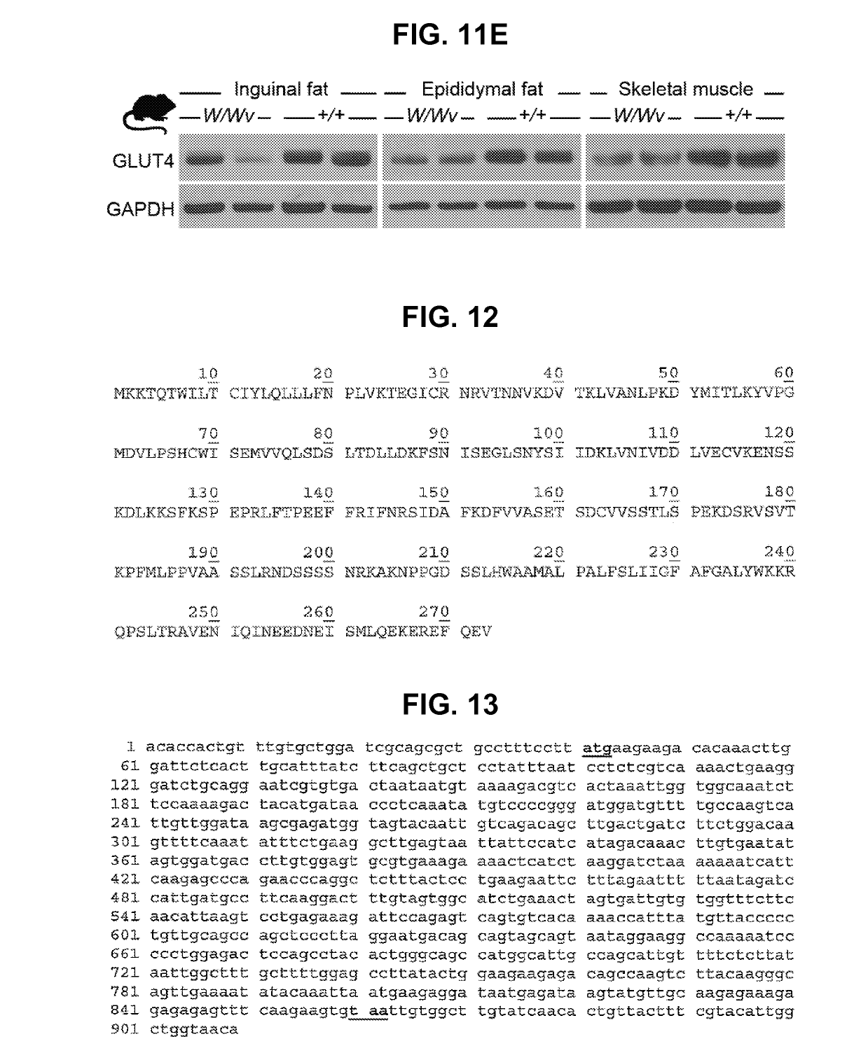

Compared with wild-type controls, KitW/KitWν mice demonstrated a greater inguinal fat mass when adjusted for body weight (FIGS. 11A,B). Whereas there was also a trend for a greater adjusted epididymal fat mass, adjusted liver mass did not differ between KitW/KitWν mice and wild-type controls (FIG. 11C-D). Considering the pivotal role for GLUT4 in systemic glucose homeostasis and insulin sensitivity, we measured GLUT4 protein expression in adipose tissue and skeletal muscle of these mice. Compared with wild-type controls, GLUT4 expression was attenuated in inguinal (subcutaneous) fat, epididymal (visceral) fat, and gastrocnemius muscle from KitW/KitWν mice (FIG. 11E). This shows that SCF and c-Kit interaction is required for GLUT4 expression in adipose tissue and skeletal muscle in vivo.

It should be understood that the examples and embodiments described herein are for illustrative purposes only and that various modifications or changes in light thereof will be suggested to persons skilled in the art and are to be included within the spirit and purview of this application

I claim:

1. A method for increasing cellular glucose uptake wherein said method comprises administering, to a subject in need of increased cellular glucose uptake, a stem cell factor (SCF) protein.

2. The method, according to claim 1, wherein said SCF protein comprises all or a portion of the sequence shown in FIG. 12 and wherein, if the SCF protein comprises less than the full sequence shown in FIG. 12, it retains the ability to stimulate cellular glucose uptake.

3. The method, according to claim 1, wherein the subject is a human.

4. The method, according to claim 1, wherein the subject has been diagnosed with a condition selected from the group consisting of insulin resistance, type 1 diabetes, type II diabetes, hyperglycemia, and metabolic syndrome.

5. The method, according to claim 4, wherein the subject has been diagnosed with type II diabetes.

6. The method, according to claim 1, wherein, as a result of the administration of the SCF protein, the expression of glucose transporter 4 (GLUT 4) is increased.

7. The method, according to claim 1, which further comprises administering to the subject an additional agent that increases cellular glucose uptake.

8. The method, according to claim 7, wherein the additional agent is selected from the group consisting of biguanides, insulin, and statins.

9. The method, according to claim 1, wherein the SCF protein is expressed from a recombinant cell that has been administered to a location at which enhanced glucose uptake is desired.

10. A method for the treatment of a condition that can be treated by activation of AMP-activated protein kinase, wherein said method comprises administering, to a subject in need of such treatment, an SCF protein.

11. The method, according to claim 10, wherein said SCF protein comprises all or a portion of the sequence shown in FIG. 12 and wherein, if the SCF protein comprises less than the full sequence shown in FIG. 12, it retains the ability to activate AMP-activated protein kinase.

12. The method, according to claim 10, wherein the subject is a human.

13. The method, according to claim 10, wherein the SCF protein is expressed from a recombinant cell that has been administered to a location at which activation of AMP-activated protein kinase is desired.

14. A method for inhibiting cellular glucose uptake in a subject in need of such inhibition, wherein said method comprises administering, to a subject in need of such inhibition, a compound that blocks the activity of SCF and/or blocks the c-Kit receptor.

15. The method, according to claim 14, wherein the subject is a human.

16. The method, according to claim 14, that is used to treat hypoglycemia.
